(19) United States
(12) Patent Application Publication
(10) Pub. No.: US 2006/0065600 A1
(43) Pub. Date: Mar. 30, 2006

(54) Processes for recovering oligomers of glycols and polymerization catalyst from waste streams

(76) Inventors: Hari Babu Sunkara, Hockessin, DE (US); James Richard Cavall, Newark, DE (US); William Wayne Goudie, Kennett Square, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/283,283

(22) Filed: Nov. 17, 2005

Related U.S. Application Data
(63) Continuation of application No. 10/423,363, filed on Apr. 25, 2003.

(51) Int. Cl. B01D 61/00; C07C 43/11

(52) U.S. Cl. 210/651; 210/652; 568/617; 568/621

(57) ABSTRACT

Processes for recovering and recycling oligomers and polymerization catalyst from aqueous waste streams generated during the production of polyalkylene ether glycols are provided. The processes utilize membranes; preferably reverse osmosis or nanofilter membranes. The processes can reduce waste and costs associated with the polymerization of polyalkylene ether glycols by recovery and recycling of glycols and catalysts

This application is a Continuation of U.S. patent application Ser. No. 10/423,363, filed Apr. 25, 2003, the entire disclosure of which is hereby incorporated by reference.

### FIELD OF THE INVENTION

This invention relates to membrane processes for recovering and recycling oligomers and polymerization catalyst from waste streams generated during the production of polyalkylene ether glycols.

### BACKGROUND

Polyalkylene ether glycols, including polyethylene glycol, poly(1,2-propylene ether) glycol, and polytetramethylene ether glycol, are widely used in industry for various end use applications. They are used widely as lubricants or as starting materials for preparing lubricants used in the molding of rubbers and in the treatment of fibers, ceramics and metals. They are also used as starting materials for preparing cosmetics and medicines, as starting materials or additives for water-based paints, paper coatings, adhesives, cellophane, printing inks, abrasives, and surfactants and as starting materials for preparing resins, such as alkyd resins. Polyalkylene ether glycols are also useful as soft, flexible segments in the preparation of copolymers and segmented copolymers such as polyurethanes, thermoplastic polyesters and unsaturated polyester resins.

Polyalkylene ether glycols are generally prepared by soluble acid- or base-catalyzed processes and therefore the crude polyalkylene ether glycols must be purified and freed from the acid or base contamination. For example, polytrimethylene ether glycol can be prepared by dehydration of 1,3-propanediol or by ring opening polymerization of oxetane using soluble acid catalysts. The abbreviation “PO3G” is used herein to denote polytrimethylene ether glycol. Methods for making PO3G from 1,3-propanediol are described in U.S. Patent Application Publication Nos. 2002/0007043A1 and 2002/0010374A1, all of which are incorporated herein by reference. Polytrimethylene ether glycol can also be prepared by a ring-opening polymerization of a cyclic ether, oxetane, as described in J. Polymer Sci., Polymer Chemistry Ed. 28, 429444 (1985), the disclosure of which is incorporated by reference. The crude polymer prepared from the polycondensation process is subsequently purified and the purification processes typically comprise (1) a hydrolysis step to hydrolyze the acid esters formed during the polymerization, (2) phase separation and water extraction steps to remove the soluble acid catalyst, generating an organic phase and a waste aqueous phase, (3) a base treatment of the organic phase, typically with a slurry of calcium hydroxide, to neutralize and precipitate the residual acid present, and (4) drying and filtration of the polymer to remove residual water and solids. The acid polymerization catalyst is sulfuric acid in this description.

The above multi-step process is effective in producing PO3G. However, the water washing steps not only remove the acid present but also remove the water-soluble short polyether chains. Depending on the molecular weight of the polymer, a significant amount (typically in the range of 1-20% by weight) of low molecular weight fraction can be extracted out, resulting in reduced yields of polymer. It is desirable to recover the oligomers from the water to maximize the product yields. In addition, the extracted acid from the polymer must be neutralized with a base before being discharged as solid waste. Washing is also capital intensive since it results in an aqueous stream that requires distillation of large amounts of water to recover the low molecular weight oligomers before discharge. These conventional waste treatment processes can create high capital, maintenance, and operating costs.

Membrane separation methods have been proposed for the fractionation of essentially anhydrous and solvent-free polyether glycols (e.g., by Dorai, et al. in U.S. Pat. No. 5,434,315). JP 49098898 (to Nippon Polyurethane Kogyo KK) describes a process for the use of a semipermeable cellophane membrane to purify crude polytetramethylene glycol. However, such a membrane-based procedure is impractical for commercial use with the two-phase initial product described above. Membrane separation methods have also been used for the recovery of polyether complexing agents from biochemical purification in JP 07089985 (to Higeta Shoyu KK), the concentration of ethylene glycol in the synthesis of ethylene oxide (Hoechst Aktiengesellschaft in British Patent GB 1,463,324). The use of membranes in the PO3G production process to recover oligomers and polymerization catalyst from aqueous extract is not known.

It would be advantageous if all or part of the waste low molecular weight oligomers and catalyst present in the aqueous extract and washings could be concentrated and recovered. Recovered oligomers can be reacted further with monomer to make products with desired molecular weights, and recycle of the catalyst can reduce costly disposal of catalyst as salts. In addition, it is desirable to provide an environmentally friendly process by minimizing the aqueous waste stream. The present invention is directed to these and other ends.

### SUMMARY OF THE INVENTION

One aspect of the present invention is a process for recovering and recycling polyalkylene ether glycol oligomers from aqueous wash streams generated during production of polyalkylene ether glycols comprising: (a) providing an aqueous wash stream comprising oligomeric polyalkylene ether glycol obtained from a process comprising a catalyzed polycondensation reaction of monomeric diol and subsequent hydrolysis of the resulting polymer; (b) providing a membrane device comprising a membrane having a retentate side and a permeate side; (c) contacting a feed stream comprising the aqueous wash stream with the retentate side of the membrane such that a portion of the feed stream is rejected to provide a retentate containing concentrated polyalkylene ether glycol oligomers, the retentate being substantially retained in contact with the retentate side of the membrane; (d) recovering the retentate containing concentrated polyalkylene ether glycol oligomers; and (e) recycling the retentate containing concentrated polyalkylene ether glycol to the polycondensation reaction.

In some preferred embodiments, the aqueous wash stream contains 1 to 20% by weight of the polyalkylene ether glycol oligomers and further comprises one or more polymerization catalysts contained in the retentate.

In one embodiment the membrane is a reverse osmosis membrane and has a sodium chloride rejection rate preferably at least about 95% and more preferably at least about 99% under standard conditions. Preferably, the aqueous wash stream is fed to the reverse osmosis membrane at a pressure of from about 50 to about 1,000 psi, more preferably from about 150 to about 600 psi.

In another embodiment the membrane is a nanofilter membrane and has a magnesium sulfate rejection rate preferably at least about 80%, more preferably at least about 95%, under standard conditions. Preferably, the aqueous wash stream is fed to the nanofilter membrane at a pressure of from about 50 to about 400 psi, more preferably from about 100 to about 200 psi.

In one embodiment the process comprises providing two or more membranes arranged in parallel or series.

Preferably the aqueous wash stream contains 1 to 20% by weight of the polyalkylene ether glycol oligomers and further comprises one or more polymerization catalysts that are contained in the retentate. More preferably the aqueous wash stream comprises one or more acids used as a polycondensation catalyst in the polycondensation reaction, and the acids are contained in the retentate. More preferably the aqueous wash stream comprises 0.1 to 2 wt. % acid.

In a preferred embodiment, the polyalkylene ether glycol is selected from the group consisting of polyethylene ether glycol, poly(1,2-propylene ether) glycol, polytrimethylene ether glycol and polytetramethylene ether glycol. More preferably the monomeric diol comprises 1,3-propanediol, the polyalkylene ether glycol is polytrimethylene glycol, and the aqueous wash stream comprises polytrimethylene ether glycol oligomers, preferably having a number average molecular weight less than about 500.

The contacting step can be within a batch or a continuous operation. When the operation is batch, it is preferred that the retentate containing concentrated polyalkylene ether glycol oligomers be recycled to the feed stream and subjected to at least one additional step of contacting with the membrane to further concentrate the oligomers before recycling the retentate containing concentrated polyalkylene ether glycol into the polycondensation reaction.

### BRIEF DESCRIPTION OF THE DRAWING

FIG. 1 shows a schematic representation for a process for making PO3G and recovering oligomers and acid catalyst from the process according to one embodiment of the invention.

### DETAILED DESCRIPTION

The present invention provides processes for recovering oligomers and/or acid catalyst from wash streams such as, for example, an aqueous wash stream of polytrimethylene ether glycol, using reverse osmosis or nanofilter membranes. The processes provide reduction of liquid and solid wastes, concentration of oligomers and acid, minimization of water usage, oligomer recycle to improve polymer yields, and thus lowered production costs, as compared to conventional processes for the production of polyalkylene ether glycols.

The term “oligomers”, as used herein, refers to linear ether diols, including starting monomeric diols, and cyclic ethers formed during the polymerization of glycols, having a number average molecular weight of less than about 500. The preferred glycol is 1,3-propanediol. Optionally, one or more other diols can be present, for example, when used as comonomers, as described in U.S. Patent Application Publication No. 2002/0010374A1, cited above. Comonomer diols that are suitable for use in the process include aliphatic diols such as, for example ethanediol; 2-methyl-1,3-propanediol; 2,2-dimethyl-1,3-propanediol; 2,2-diethyl-1,3 -propanediol; 2-ethyl-2-(hydroxymethyl)-1,3-propanediol; 1,6-hexanediol; 1,8-octanediol; 1,10-decanediol; isosorbide; 1,6-hexanediol; 1,7-heptanediol; 1,8-octanediol; 1,9-nonanediol; 1,10-decanediol; 1,12-dodecanediol; 3,3,4,4,5,5-hexafluoro-1,5-pentanedio-I; 2,2,3,3,4,4,5,5-octafluoro-1,6-hexanediol; 3,3,4,4,5,5,6,6,7,7,8,8,9,9,-10,10-hexadecafluoro-1,12-dodecanediol; cycloaliphatic diols such as, for example 1,4-cyclohexanediol; 1,4-cyclohexanedimethanol; and isosorbide; polyhydroxy compounds such as, for example, glycerol; trimethylolpropane; and pentaerythritol. Preferred comonomer diols include 2-methyl-1,3-propanediol; 2,2-dimethyl-1,3-propanediol; 2,2-diethyl-1,3-propanediol; 2-ethyl-2-(hydroxymethyl)-1,3-propanediol; 1,6-hexanediol; 1,8-octanediol; 1,10-decanediol; isosorbide, and mixtures derived therefrom. As used herein, the term “mixtures derived therefrom”, when used in reference to a list of, for example, compounds, includes any two or more compounds in the list.

The processes disclosed herein utilize a membrane. The term “feed” or “feed stream”, as used herein, refers to a pressurized aqueous solution contacting the membrane. The feed is the aqueous phase from the phase separation stage that, together with aqueous wash streams, is pumped into the membrane housing. The aqueous phase comprises 1-20% by weight of oligomers and 0.1-2% acid. In a single-pass procedure, the feed stream is fed once to the membrane housing. In a multiple-pass procedure, retentate or permeate can be partially or wholly returned to the feed. For example, it may be desirable to return retentate to the feed if it is desired to remove more water. It may be desirable to return retentate to the feed if it is desired to recover more oligomers and/or acid catalyst from the feed.

The term “permeate”, as used herein, refers to the fraction of the feed that passes through the membrane.

The term “retentate”, as used herein, refers to the concentrated fraction of the feed stream that is rejected by, i.e. does not pass through, the membrane. The retentate contains increased concentrations of oligomers and acid; the permeate contains reduced concentrations of oligomers and acid.

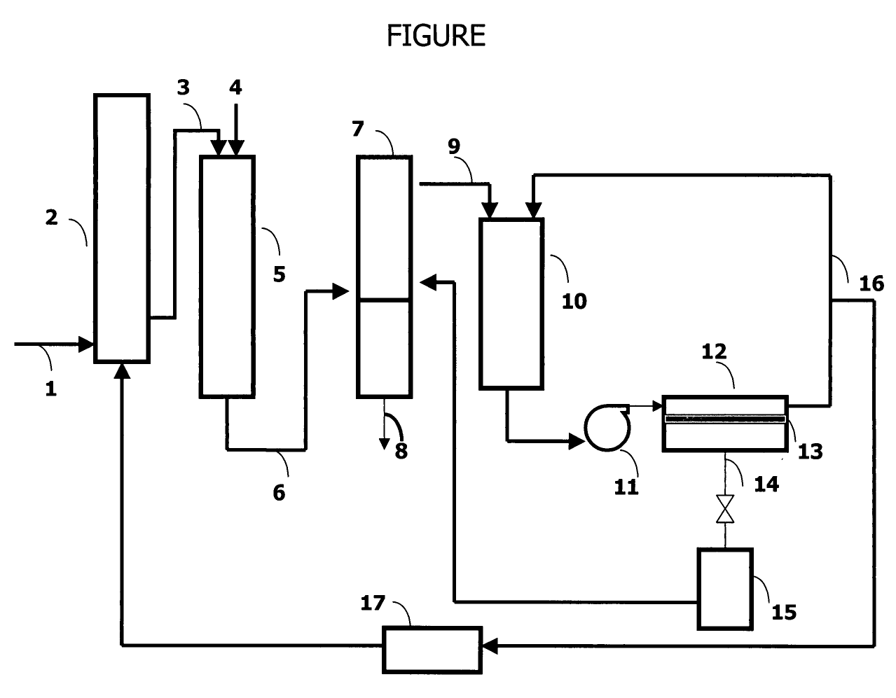

A preferred embodiment of a process for recovering oligomers is described with reference to FIG. 1. A diol and soluble acid catalyst mixture 1 is fed into a polymerization reactor 2. The polymerization reaction is continued until a desired average molecular weight is obtained. The crude polymer 3 prepared in reactor 2 containing the acid is fed into a hydrolyzer reactor 5 along with sufficient amount of water 4 to hydrolyze acid esters formed during polymerization. An aqueous polymer mixture 6, in the form of an emulsion, is fed into a settler 7 and the phases are allowed to separate into an organic (polymer) phase 8 and an aqueous phase 9. The polymer phase 8 is thoroughly washed with water and the washed polymer is processed separately. The aqueous phase and all of the collected aqueous washings 9, which comprise an aqueous solution of oligomers and acid, is fed into a feed tank 10 connected to a high-pressure pump 11. The aqueous solution is fed from the tank and circulated into a membrane housing 12 that holds membrane element 13. The membrane housing and membrane can be referred to collectively herein as a “membrane device”. As the feed materials sweep across the membrane surface, the water selectively and preferentially passes through the membrane to the permeate side of the separation unit. The permeate 14 is an aqueous solution containing a reduced concentration of oligomers and acid that is collected into a reservoir 15. The collected permeate can be recycled back to the hydrolyzer unit 5 or directed to a water washing step. The retentate 15 that comes out of the membrane housing 12 contains concentrated oligomers and acid. In order to concentrate the retentate to the desired level, multiple passes of the retentate through the separation unit are typically required. The concentrated retentate 16 can be fed into a polymerization reactor for recycling, optionally after further concentration in a water evaporator or dryer 17.

In the processes disclosed herein, both reverse osmosis or hyperfiltration (RO) and nanofilter (NF) membranes are useful, with reverse osmosis membranes being preferred. Ultrafiltration and microfiltration (microporous) membranes typically lack adequate selectivity, and are therefore not preferred. Types of membranes, measurements of their properties and performance, etc., are reviewed in Membrane Handbook, edited by W. S. Winston Ho and Kamalesh Sirkar, Van Nostrand Reinhold, New York N.Y., ISBN 0442-23747-2, 1992.

Preferred RO membranes provide a permeate containing most of the water and a retentate containing most of the oligomers and acid. Preferred RO membranes are characterized by a sodium chloride rejection rate equal or greater than 95% and preferably equal or greater than 99% under a set of standard conditions as described in Test Method 2 below. NF membranes are typically less selective to the acid catalyst than are RO membranes, allowing more acid catalyst to pass through the membrane. However, a NF membrane has a productivity (flow rate of permeate through the membrane) that is typically much higher than RO membranes, and thus may provide an advantage where productivity is more important than acid recycle. Preferred NF membranes are characterized by a magnesium sulfate rejection rate of at least about 80% and preferably at least about 95% under a set of standard conditions as described in Test Method 2 below.

Membranes can be fabricated in various shapes and produced in various assemblies. Examples include: spiral wound, in which the membrane is in the form of a flat sheet rolled into spirals with spacing materials interleaved with the membrane; tubular, in which the membrane is formed into tubes lining the inner surface of a reinforced braid, and in which the braid can be either a component in a larger tube or self-supporting and constructed of metal or ceramic; a flat sheet membrane that may be supported in a typical plate and frame structure similar to a filter press; in the form of open-ended hollow fibers organized and sealed into header plates to provide a separation of the flow over the external surfaces of the hollow fibers from any flow within the bores of the hollow fibers ensuing by virtue of passage of the liquid feed mixture across the membrane; in the form of fine tubes, typically having larger dimensions than hollow fibers and designed to permit permeate flow from the inside to the outside of the fiber; or in the form of a multi channeled monolith, in which the membrane composition is usually ceramic, and which has performance similar to fine tube design. The membrane unit used in the process can include any equipment known to those skilled in the art. Preferred are spiral wound and tubular membranes.

Preferred membrane structures are composite membranes. Composite membranes comprise a thin membrane coating on a porous support. The porous support can be fabricated from material that may not have optimal separation properties but may have desirable strength and chemical resistance properties. Another type of membrane structure that can be used is an asymmetric (anisotropic) membrane that consists essentially of a single permeable membrane material distinguished by the existence of two distinct morphological regions within the membrane structure. One region comprises a thin, relatively dense semi-permeable skin capable of selectively permeating one component of a fluid mixture. The other region comprises a less dense, porous, non-selective support region that serves to prevent the collapse of the thin skin region of the membrane under pressure.

When a preferred reverse osmosis or nanofilter is used, a preferred membrane material is a polymeric membrane that can be used in the pH range 2-11, such as, for example, membranes made from polysulfones, polyether sulfones, sulfonated polysulfones, and polyvinylidene fluoride. Cellulose acetate blend membranes can be used, although such membranes are best used in the pH range of 4-7. Frequently the composition of proprietary membranes may be unavailable. In such cases, suitable pH stability and sodium chloride or magnesium sulfate rejection tests (discussed above) can be used to screen candidate membranes. Any conventional commercially used configurations such as flat sheet; spirals or fine tube membrane can be used.

Although some of the foregoing description has been directed to specific membrane configurations, such as hollow fiber and fine tube membranes, film (flat sheet and spiral) membranes can also be used.

The preferred operation of the membrane separation process is a batch operation wherein the retentate is returned to a feed tank for recycling, which may require multiple passes of the retentate through the membrane to achieve desired concentration of oligomers and acids in water. The batch operation is generally the fastest method for concentrating a given amount of material and requires less membrane area. A single pass continuous operation can also be carried out where there is no recycling of retentate. Additionally, the reverse osmosis system can comprise one or more feed side and permeate side stages.

For use in the process, a semi-permeable membrane preferably has adequate separation properties to reject the oligomers and acid catalyst, as well as sufficient chemical resistance to the feed stream such that the membrane is not unduly or deleteriously affected by its environment in the separation process.

The retentate contains an increased concentration of the catalyst and lower molecular weight oligomers of the polyether, particularly with the use of RO membranes. The permeate contains a reduced concentration of oligomers and catalyst, particularly in the case of RO membranes. The ratio of feed volume to retentate final volume (“feed volume reduction factor”) is controlled to optimize productivity and membrane performance. The retentate, containing increased concentrations of oligomers and acid catalyst, is suitable for recycling into the initial polymerization step, wherein the oligomers undergo further polymerization. The recycled catalyst reduces the amount of fresh acid catalyst required. The permeate, containing reduced concentrations of oligomers and acid catalyst, is suitable for recycling to the hydrolysis stage. As indicated above, in order to concentrate the retentate to the desired level, multiple passes of the retentate through the separation unit are typically required.

The feed stream comprises an aqueous solution containing up to 20% by weight of low molecular weight oligomers (typically with a molecular weight of less than about 500) and up to 2% by weight of acid catalyst. The feed stream is fed to the RO membrane device at a temperature between ambient temperature (for example, about 25° C.) and 100° C. and at a pressure of 50-1000 psig (450-7000 kPa), and preferably 150-600 psig (11404240 kPa). Alternatively, the feed stream is fed to the NF membrane device at a temperature of between ambient and 100° C. and at a pressure of 50-400 psig (450-2860 kPa), and preferably 100-200 psig (790-1480 kPa). However, for any membrane, it is highly preferred that the maximum temperature and pressure do not exceed the manufacturer's specifications. Techniques for adjusting pressure, feed rate, and recycle are well known to those skilled in the art.

In one embodiment of the invention, the feed stream is subjected to filtration prior to being fed to the membrane. Filtration can remove particles and debris, which may otherwise adversely affect the membrane. The feed stream optionally can be neutralized with a base such as calcium hydroxide slurry and filtered before being fed to the membrane device when no acid recovery is desired.

### EXAMPLES

Equipment and Materials

Conductivities were measured with a Model 4P Conductivity Meter (from Myron Company, Carlsbad, Calif.), used according to the manufacturer's instructions.

FILMTEC SW30-2521 and SW 30HR-2540 seawater RO membrane elements were obtained from Dow Chemicals, Midland, Mich.

DK2540 TFM nanofilter thin-film membrane elements were purchased from Osmonics, Minnetonka, Minn.

### TEST METHODS

Test Method 1, Acid and Water Analyses

Acid analyses were performed using standard acid-base titration. The amount of water present in a feed sample is determined using a Mitsubishi Model CA-06 coulometric Karl-Fisher titrator supplied by Cosa Instrument Corp. (Norwood, N.J.). A glass gas-tight syringe is used to transfer 0.1-0.2 mL of the sample into the titration cell. Aquastar Comp-5K titration reagent containing 2-methoxyethanol, imidazole, iodine and sulfur dioxide is used. The reagent reacts rapidly and quantitatively with the water in the sample. The end point of the reaction is detected coulometrically.

Test Method 2: Membrane Characterization and Batch Concentration Test

The following procedure provides characterization of a membrane in terms of its selectivity, the ability to reject a salt in aqueous solution.

A. Initial Characterization

1. The membrane is installed in the pressure vessel according to the manufacturer's specifications.
2. The membrane is then flushed to remove any preservatives that the manufacturer may have used. The flush uses distilled water with a conductivity of less than 25 microS/cm. The retentate and permeate streams are discarded.
3. After the flush, the membrane is then characterized to determine that the membrane meets manufacture specifications.
4. The characterization is performed using sodium chloride or magnesium sulfate solution in the same quality of distilled water as used in the membrane flush. System settings are selected for the desired pressure and concentrate flow rate for the membrane under test. Preferred RO membranes are characterized by a sodium chloride rejection rate equal or greater than 95% and preferably equal or greater than 99% under a set of standard conditions. The standard conditions are 400 psig (2860 kPa) at 25° C. using a standard aqueous solution of sodium chloride in distilled water having a conductivity of 3000 Siemens per centimeter (S/cm) (corresponding to 1000-2000 mg/L NaCl). Higher pressures of 800-1000 psig (5620-7000 kPa) may be used if compatible with membrane design. The rejection rate percentage as used herein refers to the quotient of (i) the quantity of the concentration of the salt in the feed less the concentration of salt in the permeate divided by (ii) the concentration of the salt in the feed. NF membranes typically provide a lower selectivity for ionic solutes and higher productivity (flow rate of permeate through the membrane) than RO membranes, thereby providing an advantage where productivity is more important than acid recycle. NF membranes are characterized by a magnesium sulfate rejection rate of equal or greater than 80% and preferably equal or greater than 95% under a separate set of standard conditions. The standard conditions for nanofilter membranes are 400 psig (2860 kPa) at 25° C. using a standard aqueous solution of magnesium sulfate in distilled water having a conductivity of 2600 S/cm (corresponding to 1500-2500 mg/L MgSO₄). The rejection rate is calculated as for the RO membranes. Higher pressures of 800-1000 psig (5620-7000 kPa) may be used if compatible with membrane design. The procedures for determining sodium chloride and magnesium sulfate rejection rates are described in detail in the “Membrane Handbook” referenced above.
5. The following are recorded:
   Date, time, and material in the feed tank.
   Feed and concentrate pressure (psig, or kPa)
   Permeate and concentrate flow (either mL/min or gallons/min)
   Feed, concentrate, and permeate conductivities (S/cm)
   Feed temperature (° C.).
6. The five readings are recorded 5 to 15 minutes apart until the readings are constant, indicating the membrane has reached an equilibrium. Initially, the feed is high quality water, with which the initial water permeation rate is obtained.
7. The standard salt solution is then added to the feed tank. The feed pressure and concentrate flow rate are set to the desired settings. Membrane equilibrium is reestablished as in Step 6 and membrane performance recorded as in Step 5.
8. The salt solution is flushed out of the membrane with high quality water. A conductivity level below 25 microS/cm is established and feed pressure and concentrate flow are set. Readings are again taken as in step 5 to confirm that the membrane has returned to the initial water readings.
9. If the membrane performance is determined to meet the manufacturer's specifications the test proceeds.
   B. Batch Concentration Test.
10. The flush water is drained from the system and replaced with the feed material. The permeate and retentate are recirculated to the feed tank. The feed is circulated around at low pressure (20 to 60 psig, 240-515 kPa) for 10 to 20 minutes, when the system is then slowly brought up to the operating conditions for feed pressure and retentate flow rate for the test.
11. The system conditions of feed pressure and concentrate flow rate are held constant throughout the test. The system is allowed to equilibrate for 5 to 20 minutes before the same readings as in Step 5 are taken. Samples of the feed, permeate, and retentate are taken after the first or second set of readings.
12. With the system at operating conditions, a predetermined amount of material is then removed from the feed tank via the permeate system. The permeate returning to the feed tank is temporarily diverted to an empty tank while the permeate sample is collected.
13. The system is readjusted for feed pressure and concentrate flow and restabilized over 5 to 20 minutes as in Step 11.
14. Steps 12 and 13 are repeated until the final concentration level has been reached.
15. After the last set of readings, the material is drained from the system and the system flushed with high quality water. Material drained from the tank is saved for additional testing or analysis if needed.

Each example represents a fixed initial volume of feed to a selected membrane at a selected pressure. In the higher pressure Examples 1 and 3, the duration of the process is necessarily limited by the volume of the equipment, and the process is terminated prior to the point at which the remaining feed volume is no longer sufficient to prevent air being drawn into the pump. In the lower pressure Example 2 the process was terminated when the flow rate dropped below useful levels (about 0.11 cm³/s).

### Example 1

Feed Preparation

1,3-Propanediol, 13.9 kg, and 139 g concentrated sulfuric acid were added to a 22-L glass reactor and the contents polymerized at 160° C. under nitrogen until the number average molecular weight was approximated 1700. A portion (1.9 kg) of the crude polymer was transferred to a 5-L glass reactor with 1.9 kg of distilled water and the reaction mixture stirred slowly under a nitrogen blanket while heated to 100° C. After 4 hours, the heater and the stirrer were turned off and the mixture was allowed to separate into two phases by gravity. The aqueous phase was separated from the polyether phase by decantation. Distilled water (1.9 kg) was again added to the polymer phase and the reaction mixture was heated to 50° C. and then cooled. After separation into clear phases, the aqueous phase was removed from the polymer phase. The above procedure was repeated four more times and all of the collected aqueous phases were combined. A total of 5.5 gallons (20.8 L) of aqueous wash stream was obtained by repeating the hydrolysis and extraction steps as described above. The acid content of the aqueous mixture was determined by conventional acid-base titration using 0.1 N tetrabutylammonium hydroxide in methanol. The water content was measured by conventional titration with Karl Fischer reagent. The total glycol content was determined by difference.

Membrane Separation

The membrane device consisted of a feed tank, feed pump, and membrane housing that holds the membrane element. The system had instrumentation that allows for pressure, temperature, and flow control. A FILM TEC SW30HR-2540 Reverse Osmosis membrane element (diameter 61 mm, length 1016 mm, active surface area 2.8 m²) was used and the membrane separation efficiency was tested to confirm vendor specifications. This was performed using high quality water and a standard sodium chloride salt solution for characterization as described above in Test Method 2, above. Conductivity was measured to determine that the membrane has a sodium chloride rejection of greater than 99%.

The feed sample (5.5 gallons, 20.8 L) was added to the feed tank. The material was then circulated through the membrane initially at low pressure to condition the membrane. The pressure was slowly brought up to operating pressure (400 psig, 2854 kPa). The concentrate flow was set for 1.5 gpm (5.68 L/min) and the temperature was maintained between 39 and 47° C. Initial readings were taken after the system was allowed 20 minutes to equilibrate and these readings include pressure, flow, temperature, and conductivity. Feed, concentrate, and permeate samples were collected at regular intervals of time for analysis. For this test, a batch concentration was performed on the material. Initially, only the permeate stream was removed from the feed tank and collected while the concentrate continued to circulate back to the feed tank. After the appropriate amount of material has been removed, the permeate line was redirected back into the feed tank and again the system was allowed to reach equilibrium before repeating the data collection and samples. These steps were repeated until the final reduction in volume (5.5×) was reached. A final set of readings was taken along with samples and the samples were analyzed. The concentrate and overall permeate are retained. Tables 1-3 show the separation results.

TABLE 1
Membrane separation conditions and results

| | Time (min) | | | | | | | |
|---|---|---|---|---|---|---|---|---|
| | 0 | 20 | 50 | 65 | 110 | 150 | 210 | 240 |
| Permeate Flow, gpm | 0.159 | 0.159 | 0.148 | 0.143 | 0.140 | 0.137 | 0.129 | 0.108 |
| (L/min) | (0.60) | (0.60) | (0.56) | (0.54) | (0.53) | (0.52) | (0.49) | (0.41) |
| Retentate Flow, gpm | 1.50 | 1.50 | 1.50 | 1.50 | 1.50 | 1.50 | 1.50 | 1.50 |
| (L/min) | (5.68) | (5.68) | (5.68) | (5.68) | (5.68) | (5.68) | (5.68) | (5.68) |
| Volume in feed tank, | 5.5 | 5.0 | 4.5 | 3.5 | 3.0 | 2.0 | 1.5 | 1.0 |
| gallons (L) | (20.8) | (18.9) | (17.0) | (13.2) | (11.4) | (7.6) | (5.7) | (3.8) |
| Concentration factor | 1× | 1.1× | 1.2× | 1.57× | 1.83× | 2.75× | 3.6× | 5.5× |
| Feed acidity, | 56.4 | 56.7 | 66.5 | 91.8 | 95.5 | 142.6 | 172.3 | 232.7 |
| (meq/kg) | | | | | | | | |
| Permeate acidity, | 0.13 | 0.17 | 0.17 | 0.20 | 0.30 | 0.46 | 0.80 | 1.49 |
| (meq/kg) | | | | | | | | |
| % water in the feed | 97.2 | ND | 97 | ND | 96 | ND | 94.5 | 91.7 |
| % water in the | 99.1 | ND | 98.9 | ND | 99.2 | ND | 99.3 | 98.4 |
| permeate | | | | | | | | |

ND = not determined, gpm = gallons per minute

Table 1 shows that the acid concentration continuously increased in the feed. The percent water decreased continuously indicating the increase in the oligomer content in the feed. The permeate samples are mostly water and contain very little acid.

### Example 2

A FILM TEC SW30-2521 Reverse Osmosis membrane element was used and characterized as in Example 1 prior to testing, using sodium chloride solution and the procedure of Test Method 2, above. The goal was a rejection of greater than 98.5%. The 8 L feed solution was prepared as described in Example 1 except the number of water washing steps were less, resulting in higher oligomer content. This feed solution was added to the feed tank, and circulated and concentrated in the same way. For this test the material was concentrated only to (1.31×) factor and the operating pressure was 64 psig (545 kPa). The membrane separation conditions and the results are summarized and compared in Tables 2 and 3.

### Example 3

A DESAL DK-2540-1072 nanofilter membrane element was used and characterized as in Example 1 prior to testing, but using magnesium sulfate solution instead of the sodium chloride solution as in Test Method 2, above. The goal was a rejection of greater than 99%. The procedure was similar to Example 1, with 15.7 L of feed solution containing crude prepared as in Example 1 added to the feed tank, and circulated and concentrated in the same way. For this test the material was concentrated to (8.07×) factor and the operating pressure was 400 psig (2860 kPa). The membrane separation conditions and the results are summarized and compared in Tables 2 and 3.

TABLE 2
Membrane Separation Summary Results from Examples 1-3

| | Membrane type | | |
|---|---|---|---|
| | SWHR-2540A | SW30-2521-A | DK-2540-1072 |
| | Example | | |
| | 1 | 2 | 3 |
| Pressure, psig (kPa) | 400 (2860) | 64 (543) | 400 (2860) |
| Total volume | 5.5 gal (20.8 L) | 8.0 L | 15.7 L |
| Collected permeate | 4.5 gal (17.0 L) | 1.9 L | 13.8 L |
| Concentration factor | 5.5 X | 1.31 X | 8.0 X |
| Total acid in feed, g | 57.5 | 20.88 | 37.6 |
| Total acid in | 0.4 | 1.18 | 26.4 |
| permeate, g | | | |
| Oligomers in feed, | 2.8% | 8.86% | 3.7% |
| % | | | |
| Oligomers in | 8.2% | 10.5% | 11% |
| retentate, % | | | |
| Productivity, gpm* | 0.05-0.09 | 0.00182-0.00651 | 0.60-0.98 |
| (cm³/s) at 25° C. | | | |
| and at pressure | (3.15-5.7) | (0.11-0.41) | (37.9-61.8) |
| shown above. | | | |

*gpm: gallons per minute.

Table 3 below shows the same result as Table 2, but recalculated to show concentrations and distributions of components in feed, permeate, and retentate.

TABLE 3
Composition of All Streams and Distribution of Components

| | Example | | |
|---|---|---|---|
| | 1 | 2 | 3 |
| | Type | | |
| | SWHR-2540A | SW30-2521-A | DK-2540-1072 |
| Pressure, psig | 400 (2860) | 64 (543) | 400 (2860) |
| (kPa) | | | |

Volume mL (weight* %)

| | | | |
|---|---|---|---|
| Feed | 20820 (100%) | 8000 (100%) | 15700 (100%) |
| Permeate | 17034 (81.8%) | 1900 (23.7%) | 13800 (87.9%) |
| Retentate | 3785 (18.2%) | 6100 (76.3%) | 1900 (12.1%) |
| Concentration | 5.5 | 1.311475 | 8.2631579 |
| factor | | | |

Acid g (% by weight* of total acid)

| | | | |
|---|---|---|---|
| Feed | 57.5 (100.0%) | 20.9 (100.0%) | 37.6 (100.0%) |
| Permeate | 0.4 (0.7%) | 1.2 (5.7%) | 26.4 (70.2%) |
| Retentate | 57.1 (99.3%) | 19.7 (94.3%) | 11.2 (29.8%) |

Oligomer g (% by weight* of total oligomer)

| | | | |
|---|---|---|---|
| Feed | 583 (100.0%) | 709 (100.0%) | 581 (100.0%) |
| Permeate | 273 (46.8%) | 68 (9.6%) | 372 (64.0%) |
| Retentate | 310 (53.2%) | 641 (90.4%) | 209 (36.0%) |

Water g (% by weight* of total water)

| | | | |
|---|---|---|---|
| Feed | 20179 (100.0%) | 7270 (100.0%) | 15082 (100.0%) |
| Permeate | 16761 (83.1%) | 1831 (25.2%) | 13402 (88.9%) |
| Retentate | 3418 (16.9%) | 5440 (74.8%) | 1680 (11.1%) |

Concentration of acid in streams (% by weight*)

| | | | |
|---|---|---|---|
| Feed | 0.28% | 0.26% | 0.24% |
| Permeate | 0.002% | 0.063% | 0.19% |
| Retentate | 1.51% | 0.32% | 0.59% |

Concentration of oligomer in streams (% by weight*)

| | | | |
|---|---|---|---|
| Feed | 2.80% | 8.86% | 3.70% |
| Permeate | 1.82% | 3.58% | 0.19% |
| Retentate | 8.19% | 10.51% | 11.00% |

*Feed, permeate and retentate streams assumed to have density = 1000 g/L

Tables 2 and 3 indicate that the 17 L permeate sample of Example 1 shows that most of the acid retained in the retentate and thus proves the efficiency of the membrane in recovering both the acid and the oligomers. The SW30 membrane used in example 2 is also effective in separating both the acid and the oligomers. Example 3 demonstrates that the DK membrane is very effective to recover only the oligomer but not the acid and the membrane has much higher productivity than the system in example 1. The performance of each membrane is reflected in the “concentration of oligomer” and “concentration of acid” in Table 3. Productivity of the membranes is shown in Table 2. Example 1 provides the largest increase in acid and oligomer concentrations (from 0.28% to 1.51% or 5.4 times from 2.8% to 8.19% or 2.9 times, respectfully), followed by Example 3. Example 2 shows operation at a lower pressure.

What is claimed is:

1. A process for recovering and recycling polyalkylene ether glycol oligomers from aqueous wash streams generated during production of polyalkylene ether glycols comprising:
(a) providing an aqueous wash stream comprising oligomeric polyalkylene ether glycol obtained from a process comprising a catalyzed polycondensation reaction of monomeric diol and subsequent hydrolysis of the resulting polymer;
(b) providing a membrane device comprising a membrane having a retentate side and a permeate side;
(c) contacting a feed stream comprising the aqueous wash stream with the retentate side of the membrane such that a portion of the feed stream is rejected to provide a retentate containing concentrated polyalkylene ether glycol oligomers, the retentate being substantially retained in contact with the retentate side of the membrane;
(d) recovering the retentate containing concentrated polyalkylene ether glycol oligomers; and
(e) recycling the retentate containing concentrated polyalkylene ether glycol to the polycondensation reaction.

2. The process of claim 1 wherein the aqueous wash stream contains 1 to 20% by weight of the polyalkylene ether glycol oligomers and further comprises one or more polymerization catalysts, and wherein said catalysts are substantially contained in the retentate.

3. The process of claim 1 wherein said membrane is a reverse osmosis membrane and has a sodium chloride rejection rate of at least about 95% under standard conditions.

4. The process of claim 1 wherein said membrane is a reverse osmosis membrane and has a sodium chloride rejection rate of at least about 99% under standard conditions.

5. The process of claim 1 wherein said membrane is a nanofilter membrane and has a magnesium sulfate rejection rate of at least about 80% under standard conditions.

6. The process of claim 1 wherein said membrane is a nanofilter membrane and has a magnesium sulfate rejection rate of at least about 95% under standard conditions.

7. The process of claim 1 wherein said membrane is a reverse osmosis membrane and the aqueous wash stream is fed to the reverse osmosis membrane at a pressure of from about 50 to about 1,000 psi.

8. The process of claim 1 wherein said membrane is a reverse osmosis membrane and the aqueous wash stream is fed to the reverse osmosis membrane at a pressure of from about 150 to about 600 psi.

9. The process of claim 1 wherein said membrane is a nanofilter membrane and the aqueous wash stream is fed to the nanofilter membrane at a pressure of from about 50 to about 400 psi.

10. The process of claim 1 wherein said membrane is a nanofilter membrane and the aqueous wash stream is fed to the nanofilter membrane at a pressure of from about 100 to about 200 psi.

11. The process of claim 1 wherein the polyalkylene ether glycol is selected from the group consisting of polyethylene ether glycol, poly(1,2-propylene ether) glycol, polytrimethylene ether glycol and polytetramethylene ether glycol.

12. The process of claim 1 wherein the monomeric diol comprises 1,3-propanediol and the aqueous wash stream comprises polytrimethylene ether glycol oligomers.

13. The process of claim 11 wherein the polyalkylene ether glycol comprises polytrimethylene ether glycol prepared from acid catalyzed polycondensation of 1,3-propanediol, and the polyalkylene ether glycol oligomers comprise polytrimethylene ether glycol oligomers.

14. The process of claim 12 wherein the aqueous wash stream further comprises linear, cyclic, or linear and cyclic polyalkylene ether diol oligomers derived from the polycondensation of one or more monomeric diols or polyols selected from the group consisting of ethanediol, 1,6-hexanediol, 1,7-heptanediol, 1,8-octanediol, 1,9-nonanediol, 1,10-decanediol, 1,12-dodecanediol, 3,3,4,4,5,5-hexafluoro-1,5-pentanedio-I, 2,2,3,3,4,4,5,5-octafluoro-1,6-hexanediol, 3,3,4,4,5,5,6,6,7,7,8,8,9,9,-10,10-hexadecafluoro-1,12-dodecanediol, cycloaliphatic diols, 1,4-cyclohexanediol, 1,4-cyclohexanedimethanol, isosorbide, glycerol, trimethylolpropane, and pentaerythritol.

15. The process of claim 13 wherein the aqueous wash stream comprises one or more acids used as a polycondensation catalyst in the polycondensation process and wherein the one or more acids are contained in the retentate.

16. The process of claim 15 wherein said aqueous wash stream comprises 0.1 to 2 wt. % acid.

17. The process of claim 13 wherein the aqueous wash stream comprises 1 to 20 wt. % polytrimethylene ether glycol oligomers.

18. The process of claim 13 wherein the polytrimethylene ether glycol oligomers have a number average molecular weight of less than about 500.

19. The process of claim 1 wherein the process comprises providing two or more membranes arranged in parallel or series.

20. The process of claim 1 wherein the contacting step is a batch operation.

21. The process of claim 20 wherein the retentate containing concentrated polyalkylene ether glycol oligomers is recycled to the feed stream for at least one additional step of contacting with the membrane to further concentrate the oligomers before recycling the retentate containing concentrated polyalkylene ether glycol to the polycondensation reaction.

22. The process of claim 1 wherein the contacting step is a continuous operation.

23. The process of claim 18 wherein the membrane is selected from reverse osmosis membranes having a sodium chloride rejection rate of al least about 95% under standard conditions and nanofilter membranes having a magnesium sulfate rejection rate of at least about 95% under standard conditions,
wherein the aqueous wash stream comprises one or more acids used as a polycondensation catalyst in the production of polyalkylene ether glycol, and
further wherein when the membrane is a reverse osmosis membrane the aqueous wash stream is fed to the membrane at a pressure from about 150 to about 600 psi, and when the membrane is a nanofilter membrane the wash stream is fed to the membrane at a pressure from about 50 to about 400 psi.

24. The process of claim 23 wherein (i) the aqueous wash stream is obtained from a process comprising preparing polytrimethylene ether glycol by acid catalyzed polycondensation of 1,3-propanediol, (ii) the aqueous wash stream comprises one or more acids used as a polycondensation catalyst in the process, and (iii) the one or more acids is contained in the retentate.
